(19) United States
(12) Patent Application Publication
(10) Pub. No.: US 2026/0232998 A1
(43) Pub. Date: Aug. 13, 2026

(54) IMPLANTABLE STIMULATION ASSEMBLY WITH TWISTING-RESISTANT STRUCTURE

(71) Applicant: Cochlear Limited, Macquarie University, NSW (AU)

(72) Inventors: Nicholas Charles Kendall Pawsey, North Ryde (AU); Shahram Manouchehri, Pemulwuy (AU); Peter Gibson, South Coogee (AU); Charles Roger Aaron Leigh, North Epping (AU)

(21) Appl. No.: 19/161,191

(22) PCT Filed: Feb. 20, 2024
(86) PCT No.: PCT/IB2024/051581

§ 371 Date: Aug. 29, 2025

Related U.S. Application Data
(60) Provisional application No. 63/491,175, filed on Mar. 20, 2023.

(51) Int. Cl. A61N 1/05; A61N 1/372

(52) U.S. Cl. CPC A61N 1/0541; A61N 1/372

(57) ABSTRACT

An apparatus includes a stimulation assembly configured to be implanted on or within a recipient. The stimulation assembly includes an elongate body having a longitudinal axis. The stimulation assembly further includes at least one fin extending in a longitudinal direction substantially parallel to the longitudinal axis and extending from an outer surface of the body. The stimulation assembly further includes at least one stimulation element facing outwardly from the body. The at least one stimulation element is configured to be in operative communication with a portion of tissue of the recipient.

### BACKGROUND

### Field

The present application relates generally to implantable medical devices comprising stimulation arrays and systems and methods for implanting the stimulation arrays.

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, an apparatus comprises a stimulation assembly configured to be implanted on or within a recipient. The stimulation assembly comprises an elongate body having a longitudinal axis. The stimulation assembly further comprises at least one fin extending in a longitudinal direction substantially parallel to the longitudinal axis and extending from an outer surface of the body. The stimulation assembly further comprises at least one stimulation element facing outwardly from the body. The at least one stimulation element is configured to be in operative communication with a portion of tissue of the recipient.

In another aspect disclosed herein, a method comprises accessing an assembly comprising an insertion tube containing a flexible portion of an implantable device. The flexible portion has a substantially straight shape within the insertion tube and a coil shape outside the insertion tube. The implantable device comprises at least one protrusion extending outward from the flexible portion. The insertion tube comprises at least one guide channel extending along the insertion tube and at least partially containing the at least one protrusion. The method further comprises inserting the insertion tube containing the flexible portion into a region of a recipient's body. The method further comprises sliding the at least one protrusion along the at least one guide channel such that the flexible portion extends out of the insertion tube through a distal end of the insertion tube.

In another aspect disclosed herein, an apparatus comprises an elongate assembly configured to be implanted on or within a recipient. The assembly comprises a body having a longitudinal axis, at least one stimulation element facing outwardly from the body, and at least one protrusion extending from an outer surface of the body. The at least one stimulation element is configured to be in operative communication with a portion of the recipient's tissue. The at least one protrusion comprises at least one substance and is configured to release the at least one substance into the recipient's tissue.

In another aspect disclosed herein, a method comprises fabricating a stimulation assembly configured to be implanted on or within a recipient's tissue. The stimulation assembly is configured to have a flexed and substantially straight shape within a substantially straight insertion tube and to have an unflexed coil shape outside the insertion tube. Fabricating the stimulation assembly comprises providing an elongate flexible body comprising at least one stimulation element facing outwardly from the body. Fabricating the stimulation assembly further comprises forming at least one protrusion extending outward from an outer surface of the body, the at least one protrusion comprising at least one substance and configured to release the at least one substance into the recipient's tissue.

In another aspect disclosed herein, an apparatus comprises a stimulation assembly having an unflexed coil shape. The stimulation assembly comprises at least one stimulation element facing a first direction and at least one ridge extending along a length of the stimulation assembly and in a second direction different from the first direction. The apparatus further comprises a substantially straight insertion tube comprising an inner volume configured to contain the stimulation assembly flexed to have a substantially straight shape.

In another aspect disclosed herein, an apparatus comprises a stimulation electrode array comprising a body with a pre-curved configuration configured to be flexed into a substantially straight configuration prior to being implanted at least partially within a cochlea of a recipient and to return to the pre-curved configuration upon being implanted within the cochlea. The stimulation electrode array comprises a plurality of stimulation electrodes distributed along a first length of the body and configured to face modiolar wall portions of the cochlea upon being implanted within the cochlea. The apparatus further comprises at least one fin extending longitudinally along the body and outwardly from the body. The at least one fin is configured to extend away from the modiolar wall portions of the cochlea upon being implanted within the cochlea. The at least one fin is distributed along a second length of the body, the second length at least partially co-extensive to the first length. The apparatus further comprises a substantially straight insertion tube comprising an elongate channel extending longitudinally along the tube. The channel is configured to engage with the at least one fin and to allow the at least one fin to be moved along the channel during implantation of the stimulation electrode array within the cochlea. The insertion tube is sufficiently rigid to flex the stimulation assembly into the substantially straight configuration.

### BRIEF DESCRIPTION OF THE DRAWINGS

Implementations are described herein in conjunction with the accompanying drawings, in which:

FIG. 1 is a perspective view of an example auditory prosthesis implanted in a recipient with a stimulation assembly inserted into the cochlea in accordance with certain implementations described herein;

FIG. 2 schematically illustrates a simplified side view of an example internal component of an auditory prosthesis in accordance with certain implementations described herein;

FIG. 3 is cross-sectional view of the cochlea illustrating a stimulating assembly partially implanted therein in accordance with certain implementations described herein;

FIGS. 4A-4F schematically illustrate various configurations during an example implantation of a perimodiolar stimulation assembly into the cochlea of the recipient in accordance with certain implementations described herein;

FIGS. 5A-5F schematically illustrate a portion of an example apparatus comprising a stimulation assembly with at least one fin configured to be implanted on or within a recipient in accordance with certain implementations described herein;

FIG. 6A schematically illustrates a side view of an example stimulation assembly having a single contiguous fin in accordance with certain implementations described herein;

FIG. 6B is a micrograph of a side view of another example stimulation assembly having a single contiguous fin in accordance with certain implementations described herein;

FIG. 6C schematically illustrate side views of an example stimulation assembly having a plurality of fins distributed along the longitudinal axis in accordance with certain implementations described herein;

FIG. 6D schematically illustrates a cross-sectional micrograph of a cochlea overlayed with a schematically illustrated example stimulation assembly implanted within the cochlea in accordance with certain implementations described herein;

FIGS. 7A and 7B schematically illustrate cross-sectional views in a plane substantially perpendicular to the longitudinal axis of two example stimulation assemblies in accordance with certain implementations described herein;

FIGS. 8A and 8B schematically illustrate cross-sectional views in a plane substantially perpendicular to the longitudinal axis of two examples of an apparatus comprising a sheath and a stimulation assembly in accordance with certain implementations described herein;

FIG. 9A schematically illustrates a top view of a portion of another example apparatus in accordance with certain implementations described herein;

FIG. 9B is a micrograph of a top view of the portion of the example apparatus schematically illustrated by FIG. 9A;

FIGS. 9C and 9D schematically illustrate cross-sectional views in a plane substantially perpendicular to the longitudinal axis of two examples of an apparatus comprising a sheath and a stimulation assembly with a buckled fin in accordance with certain implementations described herein;

FIGS. 10A-10C schematically illustrate finite element simulations of a portion of an example pre-curved stimulation assembly in various configurations in accordance with certain implementations described herein; and

FIG. 11 is a flow diagram of an example method in accordance with certain implementations described herein.

### DETAILED DESCRIPTION

Certain implementations described herein provide a pre-curved stimulation assembly configured to be implanted into the recipient's body (e.g., into the recipient's cochlea) via a substantially straight sheath of an insertion device. The stimulation assembly comprises at least one protrusion (e.g., fin; ridge; strip) extending along a longitudinal axis of the stimulation assembly and extending outwardly from the stimulation assembly in a direction substantially perpendicular to the longitudinal axis. The stimulation assembly is in a substantially straight configuration prior to and during the implantation process and in a curved configuration upon being implanted. The at least one protrusion can be configured to engage with a corresponding at least one guide channel of the sheath to resist (e.g., prevent; inhibit) the stimulation assembly from twisting around the longitudinal axis prior to and during the implantation process. The at least one protrusion can be further configured to be compressed in a direction substantially parallel to the longitudinal axis by the straightening deformation (e.g., when the stimulation assembly is constrained; within the sheath) and to provide a restoring force (e.g., in a direction opposite to the straightening deformation of the stimulation assembly) that counters the straightening deformation of the stimulation assembly and curves the stimulation assembly (e.g., when the stimulation assembly is not constrained; outside the sheath).

The teachings detailed herein are applicable, in at least some implementations, to any type of implantable medical device (e.g., implantable stimulation system) comprising a first portion implanted on or within the recipient's body and configured to provide stimulation signals to a portion of the recipient's body and a second portion (e.g., implanted on or within the recipient or external to the recipient's body) configured to provide control signals to the first portion. For example, the implantable medical device can comprise a sensor (e.g., auditory) prosthesis system, a neurostimulation system, or a muscle stimulation system. Implementations can include any type of medical device that can utilize the teachings detailed herein and/or variations thereof.

Merely for ease of description, apparatus and methods disclosed herein are primarily described with reference to an illustrative medical device, namely a cochlear implant. However, the teachings detailed herein and/or variations thereof may also be used with a variety of other medical devices that provide a wide range of therapeutic benefits to recipients, patients, or other users. In some implementations, the teachings detailed herein and/or variations thereof can be utilized in other types of implantable medical devices beyond auditory prostheses. For example, apparatus and methods disclosed herein and/or variations thereof may also be used with one or more of the following: vestibular devices (e.g., vestibular implants); visual devices (e.g., bionic eyes); visual prostheses (e.g., retinal implants); sensors; cardiac pacemakers; drug delivery systems; defibrillators; functional electrical stimulation devices; catheters; brain implants; seizure devices (e.g., devices for monitoring and/or treating epileptic events); sleep apnea devices; electroporation; pain relief devices; swallowing treatment devices (e.g., devices for treating difficulties with the hyoglossus and/or thyrohyoid muscles); dysphagia treatment devices; devices for treating dry mouth (e.g., xerostomia or hyposalivation), devices for treating excessive or absence of muscle movement due to stroke, Parkinson's disease, or other brain disorders, devices for treating hypertension (e.g., by stimulating the carotid sinus barosensory system); etc.

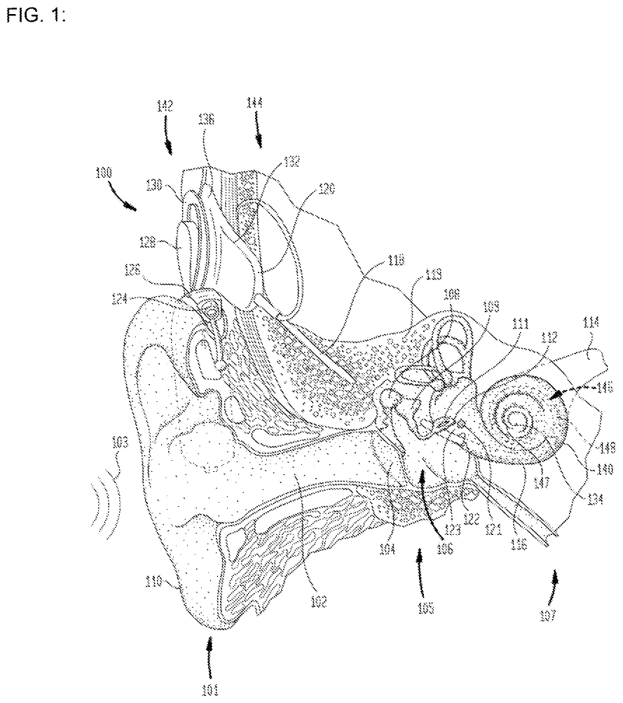

FIG. 1 is a perspective view of an example auditory prosthesis 100 (e.g., cochlear implant), implanted in a recipient with a stimulation assembly 118 inserted into the cochlea 140 in accordance with certain implementations described herein. As shown in FIG. 1, the recipient has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within the cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1, the example auditory prosthesis 100 comprises one or more components which are temporarily or permanently implanted in the recipient. The example auditory prosthesis 100 is shown in FIG. 1 with an external component 142 which is directly or indirectly attached to the recipient's body, and an internal component 144 which is temporarily or permanently implanted in the recipient (e.g., positioned in a recess of the temporal bone adjacent to the auricle 110 of the recipient). The external component 142 typically comprises one or more sound input elements (e.g., an external microphone 124) for detecting sound, a sound processing unit 126 (e.g., disposed in a Behind-The-Ear unit), a power source (not shown), and an external transmitter unit 128. In the illustrative implementation of FIG. 1, the external transmitter unit 128 comprises an external coil 130 (e.g., a wire antenna coil comprising multiple turns of electrically insulated single-strand or multi-strand platinum or gold wire) and, preferably, a magnet (not shown) secured directly or indirectly to the external coil 130. The external coil 130 of the external transmitter unit 128 is part of an inductive radio frequency (RF) communication link with the internal component 144. The sound processing unit 126 processes the output of the microphone 124 that is positioned externally to the recipient's body, in the depicted implementation, by the recipient's auricle 110. The sound processing unit 126 generates encoded signals, sometimes referred to herein as encoded data signals, which are provided to the external transmitter unit 128 (e.g., via a cable).

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate stimulation assembly 118. In some implementations, the internal receiver unit 132 and the stimulator unit 120 are hermetically sealed within a biocompatible housing, sometimes collectively referred to as a stimulator/receiver unit. The internal receiver unit 132 comprises an internal coil 136 (e.g., a wire antenna coil comprising multiple turns of electrically insulated single-strand or multi-strand platinum or gold wire), and preferably, a magnet (also not shown) fixed relative to the internal coil 136. The internal coil 136 receives power and/or data signals from the external coil 130 via a transcutaneous energy transfer link (e.g., an inductive RF link). The stimulator unit 120 generates electrical stimulation signals based on the data signals, and the stimulation signals are delivered to the recipient via the elongate stimulation assembly 118.

The elongate stimulation assembly 118 has a proximal end connected to the stimulator unit 120, and a distal end implanted in the cochlea 140. The stimulation assembly 118 extends from the stimulator unit 120 to the cochlea 140 through the mastoid bone 119. In some implementations, the stimulation assembly 118 may be implanted at least in the basal region 116, and sometimes further. For example, the stimulation assembly 118 may extend towards the apical end of the cochlea 140, referred to as the cochlea apex 134. In certain circumstances, the stimulation assembly 118 may be inserted into the cochlea 140 via a cochleostomy 122. In other circumstances, a cochleostomy 122 may be formed through the round window 121, the oval window 112, the promontory 123, or through an apical turn 147 of the cochlea 140.

The elongate stimulation assembly 118 comprises a longitudinally aligned and distally extending array 146 (e.g., electrode array; contact array) of stimulation elements 148 (e.g., electrical electrodes; electrical contacts; optical emitters; optical contacts). For example, the stimulation elements 148 can comprise intra-cochlear electrodes (ICEs) and/or extra-cochlear electrodes (ECEs). The stimulation elements 148 are longitudinally spaced from one another along a length of the elongate body of the stimulation assembly 118. For example, the stimulating assembly 118 can comprise an array 146 comprising twenty-two (22) stimulation elements 148 that are configured to deliver stimulation to the cochlea 140. Although the array 146 of stimulation elements 148 can be disposed on the stimulation assembly 118, in most practical applications, the array 146 is integrated into the stimulation assembly 118 (e.g., the stimulation elements 148 of the array 146 are disposed in the stimulation assembly 118). As noted, the stimulator unit 120 generates stimulation signals (e.g., electrical signals; optical signals) which are applied by the stimulation elements 148 to the cochlea 140, thereby stimulating the auditory nerve 114.

While FIG. 1 schematically illustrates an auditory prosthesis 100 utilizing an external component 142 comprising an external microphone 124, an external sound processing unit 126, and an external power source, in certain other implementations, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

A variety of types of intra-cochlear stimulation assemblies 118 are compatible with certain implementations described herein, including but not limited to: short, straight, and perimodiolar. A perimodiolar stimulation assembly 118 is configured to adopt a curved configuration during and/or after implantation into the cochlea 140. To achieve this, in certain implementations, the perimodiolar stimulation assembly 118 is pre-curved to the same general curvature of the cochlea 140. Such examples of the stimulation assembly 118 can be held straight by, for example, a stiffening stylet (not shown in FIG. 1) or sheath 260 which is removed during implantation, or alternatively varying material combinations or the use of shape memory materials, so that the stimulation assembly 118 may adopt its curved configuration when in the cochlea 140. Other methods of implantation, as well as other stimulation assemblies 118 which adopt a curved configuration, may be used. The stimulation assembly 118 of certain other implementations comprises a non-perimodiolar stimulation assembly 118. For example, the stimulation assembly 118 can comprise a straight stimulation assembly 118 or a mid-scala assembly which assumes a mid-scala position during or following implantation. Alternatively, the stimulation assembly 118 can comprise a short electrode implanted into at least the basal region of the cochlea 140. The stimulation assembly 118 can extend towards the apical end of the cochlea 140, referred to as the cochlea apex. In certain implementations, the stimulation assembly 118 is configured to be inserted into the cochlea 140 via a cochleostomy (e.g., formed through the oval window 112, the round window 121, the promontory 123, or through an apical turn of the cochlea 140).

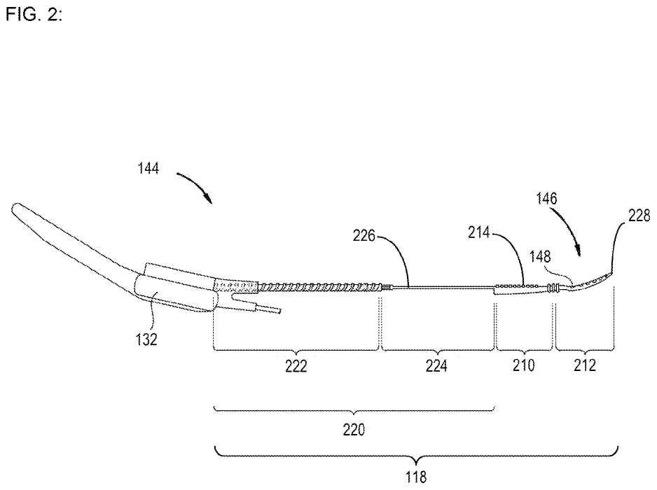

FIG. 2 schematically illustrates a simplified side view of an example internal component 144 comprising an internal receiver unit 132 which receives encoded signals from an external component 142 of the auditory prosthesis 100 (e.g., cochlear implant system). The internal component 144 terminates in the stimulation assembly 118 that comprises an extra-cochlear region 210 and an intra-cochlear region 212. The intra-cochlear region 212 is configured to be implanted in the recipient's cochlea 140 and has disposed thereon the longitudinally aligned and distally extending array 146 (e.g., electrode array; contact array) comprising a plurality of stimulation elements 148. In the example schematically illustrated in FIG. 2, the plurality of stimulation elements 148 comprises electrical contacts (e.g., electrodes) configured to apply electrical stimulation and/or optical contacts (e.g., emitters) configured to apply optical stimulation, either alone or in conjunction with electrical or other stimulation mechanisms.

In certain implementations, the stimulation assembly 118 comprises a lead region 220 coupling the internal receiver unit 132 to the array 146. In certain implementations, optical and/or electrical stimulation signals generated by the internal receiver unit 132 are delivered to the array 146 via the lead region 220. The lead region 220 comprises a first portion 222 configured to accommodate movement (e.g., is flexible) and a second portion 224 configured to connect the first portion 222 to the array 146. The first portion 222 of certain implementations is configured to prevent the stimulation assembly 118, the lead region 220 and its connection to the internal receiver unit 132, and the array 146 from being damaged due to movement of the internal component 144 (or part of the internal component 144) which may occur, for example, during mastication. In certain implementations, the second portion 224 comprises a distinct connection to the first portion 222 and/or the array 146, while in certain other implementations, the second portion 224 is blended into the first portion 222 and/or the array 146. The relative lengths of the stimulation assembly 118, the lead region 220, the first portion 222, the second portion 224, the extra-cochlear region 210, the intra-cochlear region 212, and the array 146 are not shown to scale in FIG. 2.

In certain implementations, the lead region 220 comprises a body 226 and a plurality of signal conduits (e.g., electrical wire leads; optical waveguides) (not shown) within the body 226. For example, the body 226 can comprise silicone or other biocompatible material in which the signal conduits are embedded (e.g., the body 226 is molded around the signal conduits) or the body 226 can comprise a tube in which the signal conduits are contained (e.g., the tube backfilled with silicone). The signal conduits of certain implementations comprise wires (e.g., platinum; platinum-iridium alloys) having outer diameters that are wavy or helixed around an axis substantially parallel to the longitudinal direction of the lead region 220 (e.g., within the first portion 222) and/or are substantially straight and substantially parallel to the longitudinal direction (e.g., within the second portion 224). In certain implementations, each of the signal conduits is connected to a corresponding one of the plurality of stimulation elements 148 of the array 146.

In certain implementations, the extra-cochlear region 210 is located in the middle ear cavity of the recipient after implantation of the intra-cochlear region 212 into the cochlea 140. Thus, the extra-cochlear region 210 corresponds to a middle ear cavity sub-section of the array 146. In certain implementations, an outer surface of the extra-cochlear region 210 comprises nubs 214 configured to aid in the manipulation of the stimulation assembly 118 during insertion of the intra-cochlear region 212 into the cochlea 140.

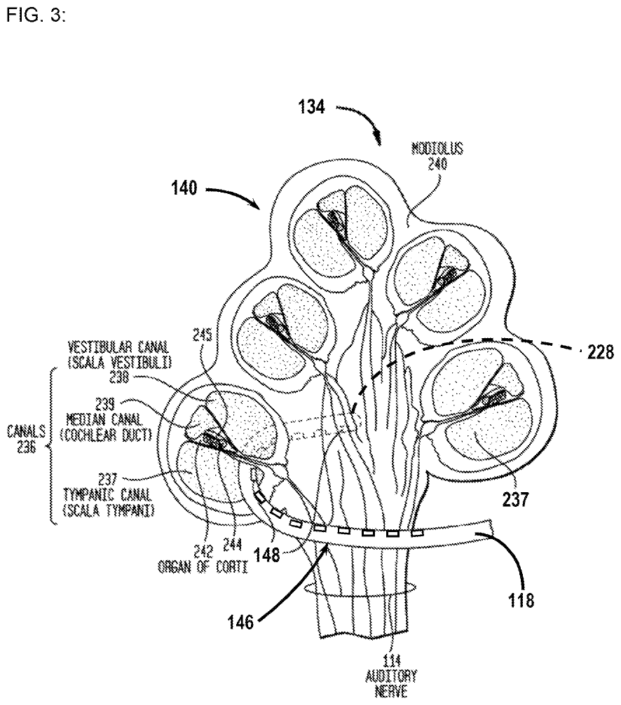

FIG. 3 is cross-sectional view of the cochlea 140 illustrating the stimulating assembly 118 partially implanted therein in accordance with certain implementations described herein. Only a subset of the stimulation elements 148 of the stimulation assembly 118 is shown in FIG. 3. The cochlea 140 is a conical spiral structure that comprises three parallel fluid-filled canals or ducts, collectively and generally referred to herein as canals 236. Canals 236 comprise the tympanic canal 237, also referred to as the scala tympani 237, the vestibular canal 238, also referred to as the scala vestibuli 238, and the median canal 239, also referred to as the scala media 239. The cochlea 140 includes the modiolus 240 which is a conical shaped central region around which the cochlea canals 236 spiral. The modiolus 240 consists of spongy bone in which the cochlea nerve cells, sometimes referred to herein as the spiral ganglion cells, are situated. The cochlea canals 236 generally turn 2.5 times around the modiolus 240.

In normal hearing, sound entering the auricle 110 (see, e.g., FIG. 1) causes pressure changes in the cochlea 140 that travel through the fluid-filled tympanic and vestibular canals 237, 238. The organ of Corti 242, which is situated on the basilar membrane 244 in scala media 239, contains rows of hair cells (not shown) which protrude from its surface. Located above the hair cells is the tectoral membrane 245 which moves in response to pressure variations in the fluid-filled tympanic and vestibular canals 237, 238. Small relative movements of the layers of the tectoral membrane 245 are sufficient to cause the hair cells to move, thereby causing the creation of a voltage pulse or action potential which travels along the associated nerve fibers that connect the hair cells with the auditory nerve 114. The auditory nerve 114 relays the impulses to the auditory areas of the brain (not shown) for processing.

Typically, in cochlear implant recipients, some portion of the cochlea 140 (e.g., the hair cells) is damaged such that the cochlea 140 cannot transduce pressure changes into nerve impulses for relay to the brain. As such, the stimulating elements 148 of the stimulating assembly 118 are used to directly stimulate the cells to create nerve impulses resulting in perception of a received sound (e.g., to evoke a hearing percept).

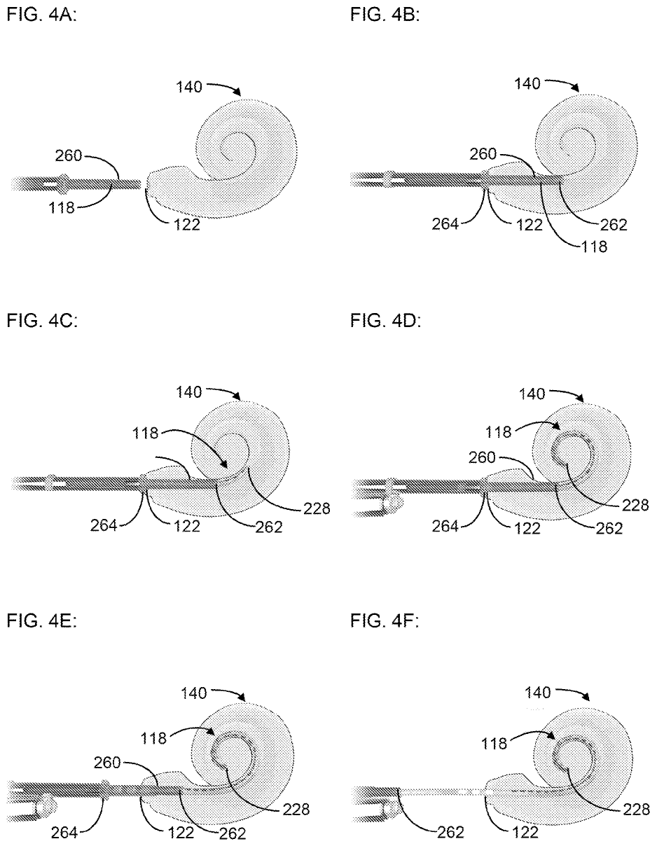

FIGS. 4A-4F schematically illustrate various configurations during an example implantation of a perimodiolar stimulation assembly 118 into the cochlea 140 of the recipient in accordance with certain implementations described herein. The perimodiolar stimulation assembly 118 is substantially enclosed in a sheath 260 (e.g., cannula; insertion tube) of an insertion tool configured to protect the stimulation assembly 118 during the implantation process. The sheath 260 is further configured to provide sufficient rigidity to maintain the pre-curved perimodiolar stimulation assembly 118 in a substantially straight configuration during at least a portion of the implantation process.

In certain implementations, the implantation process includes creating an opening (e.g., facial recess) through the recipient's mastoid bone 119 (see, e.g., FIG. 1) to access the recipient's middle ear cavity (see, e.g., FIG. 1). A cochleostomy 122 is created from the middle ear cavity into the cochlea 140 (e.g., through the round window 121, oval window 112, the promontory 123, etc. of the cochlea 140). The stimulation assembly 118 and the surrounding sheath 260 are advanced (e.g., pushed) through the opening through the mastoid bone 119 and are positioned to be inserted into the cochleostomy 122, as schematically illustrated in FIG. 4A.

As schematically illustrated in FIG. 4B, the stimulation assembly 118 and the sheath 260 are advanced (e.g., pushed) together through the cochleostomy 122 to insert a distal end portion 262 of the sheath 260 within the cochlea 140 while the stimulation assembly 118 remains in the sheath 260. The advancement of the stimulation assembly 118 and the surrounding sheath 260 is stopped once a proximal end portion 264 of the sheath 260 is in a predetermined position (e.g., in contact with the cochlea 140 at the cochleostomy 122). As schematically illustrated by FIG. 4C, the stimulation assembly 118 is then gently advanced (e.g., pushed) forward into the cochlea 140 through the sheath 260 such that an apical (e.g., distal end/tip) portion 228 of the array 146 exits the sheath 260 through an opening in the distal end portion 262 of the sheath 260. The portion of the stimulation assembly 118 that extends out of the sheath 260 is no longer constrained to be straight by the stimulation assembly 118 and therefore returns to its pre-curved configuration to follow the curvature of the canals 236 within the cochlea 140. As schematically illustrated by FIG. 4D, the advancement of the stimulation assembly 118 continues until the stimulation assembly 118 achieves the implanted position. For example, the implanted position can be the position at which the apical portion 228 of the array 146 is placed at a selected angular position (e.g., the apical portion 228 of the array 146 is at the cochlea apex 134). Once the stimulation assembly 118 achieves the implanted position, the sheath 260 can be withdrawn from the cochlea 140 (e.g., pulled out) through the cochleostomy 122, as schematically illustrated by FIGS. 4E-4F.

The effectiveness of the stimulation by the stimulation assembly 118 depends, at least in part, on the place along the basilar membrane 244 where the stimulation is delivered. That is, the cochlea 140 has characteristically been referred to as being “tonotopically mapped,” in that regions of the cochlea 140 toward the basal end are more responsive to high frequency signals, while regions of cochlea 140 toward the cochlea apex 134 are more responsive to low frequency signals. These tonotopical properties of the cochlea 140 are exploited in a cochlear implant by delivering stimulation within a predetermined frequency range to a region of the cochlea 140 that is most sensitive to that particular frequency range. However, this stimulation relies on the particular stimulation elements 148 having a final implanted positioned adjacent to a corresponding tonotopic region of the cochlea 140 (e.g., a region of the cochlea 140 that is sensitive to the frequency of sound represented by the stimulation element 148).

To achieve a selected final implanted position, the apical portion 228 of the array 146 is placed at a selected angular position (e.g., angular insertion depth; angular rotation of the apical portion 228 of the array 146 from the cochleostomy 122 through which the stimulation assembly 118 enters the cochlea 140). In certain implementations, while the stimulation assembly 118 is being implanted (e.g., during a surgical procedure conducted by an operator, such as a medical professional, surgeon, and/or an automated or robotic surgical system), a location and/or an orientation of the array 146 relative to the cochlea 140 (e.g., collectively referred to as the pose of the array 146) is adjusted as the array 146 is advanced and placed into position within the cochlea 140. The goal of the implantation is that the fully-implanted array 146 has an optimal pose in which the array 146 is positioned such that the stimulation elements 148 are adjacent to the corresponding tonotopic regions of the cochlea 140. To achieve the optimal pose, the array 146 is expected to follow a trajectory in the cochlea 140 whereby (i) the stimulation elements 148 are distributed linearly along an axis of the scala media 239, (ii) the array 146 does not make contact with the basilar membrane 244, and (iii) the stimulation elements 148 are in close proximity to the modiolar wall (e.g., if the array 146 is pre-curved) or the stimulation elements 148 are distant from the modiolar wall (e.g., if the array 146 is not pre-curved).

However, one or more these expectations may be violated during insertion of the array 146. For example, the apical portion 228 of the array 146 can become snagged on the wall of the scala media 239t, the array 146 can become buckled, folded, twisted, and/or overinserted, and/or portions of the cochlea 140 (e.g., scala tympani 237; scala vestibuli 238; scala media 239; organ of Corti 242; basilar membrane 244) can be dislocated, resulting in sub-optimal placement of the array 146. In addition, a perimodiolar stimulation assembly 118 that is pre-curved to the same general curvature of the cochlea 140 and is held straight and inserted into the cochlea 140 by a substantially straight sheath 260 can tend to twist when straightened for insertion into the cochlea 140 due to asymmetries in construction and loading. Such twisting can result in the array 146 deviating from the desired insertion trajectory within the cochlea 140, potentially resulting in foldover, scala translocation, or trauma. The tendency to twist can become more pronounced when the sheath 260 is bent in a direction opposite to the curve of the array 146 (e.g., towards the modiolar wall of the cochlea 140), as the deformation and stresses induced in the body 226 of the array 146 inside the sheath 260 are increased, thereby increasing the risk of foldover or jamming. Various structures of the sheath 260 have been disclosed to attempt to limit, reduce, or minimize the probability of such twisting (see, e.g., U.S. Pat. No. 9,713,713; U.S. Pat. Appl. Publ. No. 2020/0405351).

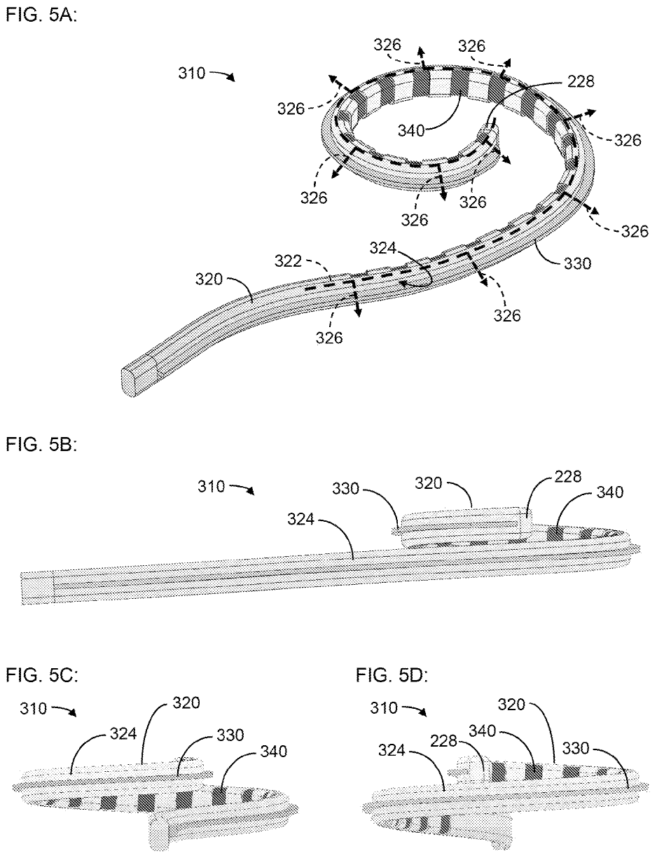

FIGS. 5A-5F schematically illustrate a portion of an example apparatus 300 comprising a stimulation assembly 310 (e.g., stimulation assembly 118; internal component 144) with at least one fin 330 configured to be implanted on or within a recipient in accordance with certain implementations described herein. FIGS. 5A-5D show various perspective views of the stimulation assembly 310, FIG. 5E shows a side view of the stimulation assembly 310, and FIG. 5F shows a cross-sectional view in a plane substantially perpendicular to a longitudinal axis 322.

The stimulation assembly 310 of FIGS. 5A-5F comprises an elongate body 320 having a longitudinal axis 322 and at least one fin 330. The at least one fin 330 extends in a longitudinal direction substantially parallel to the longitudinal axis 322 and extends from an outer surface 324 of the body 320 in at least one radial direction 326 substantially perpendicular to the longitudinal axis 322. The stimulation assembly 310 further comprises at least one stimulation element 340 facing outwardly from the body 320. The at least one stimulation element 340 is configured to be in operative communication with a portion of tissue of the recipient.

In certain implementations, as schematically illustrated by FIGS. 5A-5F, the at least one stimulation element 340 comprises an array of stimulation elements 340 distributed along the longitudinal axis 322 (e.g., array 146 of stimulation elements 148). Examples of stimulation elements 148 compatible with certain implementations described herein include but are not limited to: electrical electrodes, electrical contacts, optical emitters, and optical contacts. The stimulation elements 340 can be configured to apply stimulation signals (e.g., electrical signals; optical signals) to a portion of the recipient's body (e.g., the cochlea 140).

In certain implementations, the apparatus 300 further comprises a substantially straight sheath 260 (not shown in FIGS. 5A-5F) configured to contain the stimulation assembly 310. The sheath 260 can comprise a distal end portion 262 through which the stimulation assembly 310 is configured to be extended out of the sheath 260 (e.g., during an implantation process). The sheath 260 has sufficient rigidity to remain substantially straight and to constrain at least a portion of the stimulation assembly 310 within the sheath 260 also be substantially straight. For example, the body 320 is sufficiently flexible such that the longitudinal axis 322 of a first portion of the body 320 contained within the sheath 260 is substantially straight and the longitudinal axis 322 of a second portion of the body 320 extended out from the end of the sheath 260 is substantially curved (see, e.g., FIGS. 4A-4F).

In certain implementations, when not constrained by the sheath 260, the body 320 has a substantially curved (e.g., spiral) shape with the longitudinal axis 322 of the portion of the body 320 curved in a direction in which the at least one stimulation element 340 faces. For example, as shown in FIGS. 5A-5E, which schematically illustrate the curved configuration of the stimulation assembly 310 not constrained by the sheath 260, the stimulation elements 340 face inwardly towards a center of curvature of the body 320. The curvature of the body 320 can be configured to conform to the curvature of the canals 236 of the human cochlea 140 (e.g., the body 320 has a radius of curvature in a range of 2 millimeters to 5 millimeters).

In certain implementations, the body 320 and the at least one fin 330 comprise the same elastomeric material, while in certain other implementations the body 320 comprises a first elastomeric material and the at least one fin 330 comprises a second elastomeric material different from the first elastomeric material. Examples of elastomeric materials for the body 320 and/or the at least one fin 330 compatible with certain implementations described herein include but are not limited to: silicone rubber and other biocompatible polymers. In certain implementations, the at least one fin 330 comprises a material (e.g., hydrogel) configured to dissolve some time after implantation.

The body 320 and/or the at least one fin 330 can further comprise a biocompatible reinforcing material (e.g., fibers; mesh) embedded in the body 320 and/or the at least one fin 330, the reinforcing material configured to provide structural reinforcement and additional curved shape recovery. For example, the reinforcing material can comprise a superelastic material (e.g., shape memory alloy; nitinol), the superelastic material configured to facilitate the stimulation assembly 310 changing shape from the substantially straight configuration while the stimulation assembly 310 is within the sheath 260 to the curved (e.g., spiral) configuration upon the stimulation assembly 310 exiting the sheath 260. The reinforcing material can have a wavy (e.g., serpentine) shape that is embedded in the body 320 with peaks of the wave shape embedded within (e.g., extending inside) the at least one fin 330.

In certain implementations, the at least one fin 330 comprises a material containing at least one substance (e.g., medicament; drug; antimicrobial agent; anti-inflammatory agent; oto-protective agent; neuroregenerative agent; neurotropic agent; gene therapy agent) and that is configured to controllably introduce (e.g., release; elute) the at least one substance into the recipient's tissue. For example, the at least one fin 330 can comprise a biodegradable material having a plurality of pores containing the at least one substance, and the biodegradable material is configured to release the at least one substance as the biodegradable material degrades (e.g., dissolves) after the stimulation assembly 310 has been implanted. For another example, the at least one fin 330 can be configured to selectively release the at least one substance in response to at least one of a temperature and a moisture content of the tissue in which the stimulation assembly 310 has been implanted. The at least one fin 330 can have a sufficiently large surface area (e.g., both sides of the at least one fin 330) to release an efficacious amount of the at least one substance into the recipient's tissue. Different portions of the at least one fin 330 can contain different substances.

In certain implementations, the at least one fin 330 comprises a first portion adjacent to the body 320 can comprise silicone that does not contain the at least one substance and a second portion adjacent to the first portion and spaced from the body 320 can comprise silicone comprising the at least one substance. For example, the at least one fin 330 can be fabricated using a first molding process in which the first portion of the at least one fin 330 is affixed to the body 320 and a second molding process (e.g., subsequent to the first molding process) in which the second portion of the at least one fin 330 is affixed to the first portion. In this way, the at least one substance can be prevented from leaking over the stimulation assembly 310 during the fabrication process.

As schematically illustrated by FIG. 5F, the body 320 can have a substantially rectangular shape in the cross-sectional plane substantially perpendicular to the longitudinal axis 322 with a first width W₁ in a first direction (e.g., in a range of 0.1 millimeter to 0.5 millimeter; in a range of 0.2 millimeter to 0.4 millimeter) and a second width W₂ in a second direction substantially perpendicular to the first width (e.g., in a range of 0.2 millimeter to 0.6 millimeter; in a range of 0.3 millimeter to 0.5 millimeter). The at least one fin 330 can also have a substantially rectangular shape in the cross-sectional plane substantially perpendicular to the longitudinal axis 322 with a height H (e.g., in a range of 50 microns to 250 microns) in the first direction extending away from the outer surface 324 and a thickness T (e.g., in a range of 50 microns to 100 microns) in the second direction. In certain implementations, the at least one fin 330 has an aspect ratio (e.g., H/T) in the cross-sectional plane substantially perpendicular to the longitudinal axis 322, the aspect ratio in a range of greater than or equal to 2 (e.g., substantially equal to 3). In certain implementations, the height H of the at least one fin 330 is less than or equal to the first width W₁, while in certain other implementations the height H is greater than the first width W₁ (e.g., by more than a factor of two). Other shapes and dimensions of the body 320 and the at least one fin 330 are also compatible with certain implementations described herein.

In certain implementations, the at least one fin 330 extends along the entire length of the array of stimulation elements 340 distributed along the longitudinal axis 322, while in certain other implementations, the at least one fin 330 extends along only a portion of the array of stimulation elements 340 (e.g., only along the curved portion of the array of stimulation elements 340). For example, in certain implementations in which the at least one fin 330 comprises a single contiguous fin 330, the single contiguous fin 330 can have a length L_{F} in the longitudinal direction that is greater than or equal to a length L_{S} of the array of stimulation elements 340 in the longitudinal direction (see, e.g., FIGS. 5A-5E).

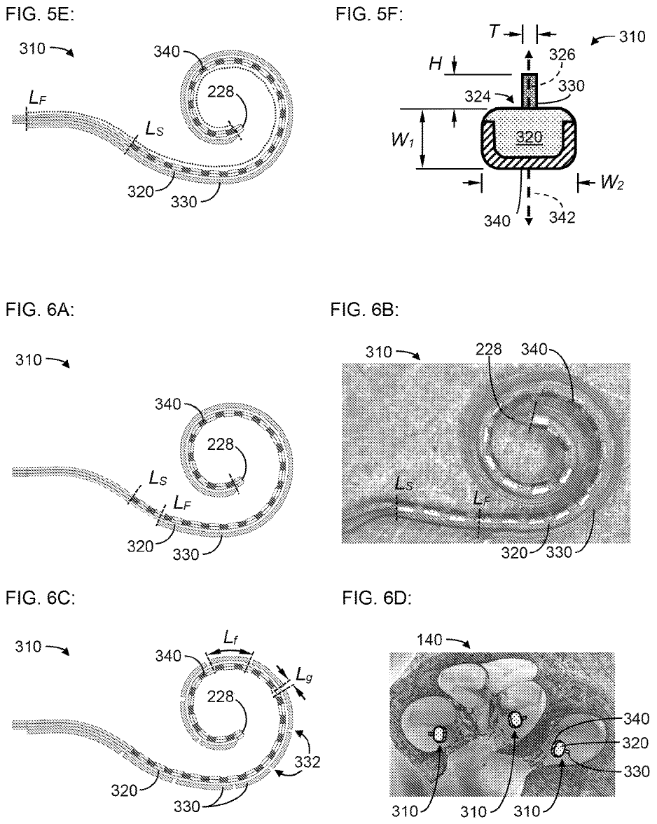

FIG. 6A schematically illustrates a side view of an example stimulation assembly 310 having a single contiguous fin 330 in accordance with certain implementations described herein. FIG. 6B is a micrograph of a side view of another example stimulation assembly 310 having a single contiguous fin 330 in accordance with certain implementations described herein. The single contiguous fins 330 of FIGS. 6A and 6B each have a length L_{F} in the longitudinal direction that is less than the length L_{S} of the array of stimulation elements 340 in the longitudinal direction (e.g., the fin 330 extends along only part of the array of stimulation elements 340). In certain implementations (e.g., as shown in FIG. 6A and FIGS. 5A-5E), the at least one fin 330 does not extend to the apical portion 228 of the stimulation assembly 310. In certain other implementations, (e.g., as shown in FIG. 6B), the at least one fin 330 does extend to the apical portion 228 of the stimulation assembly 310.

FIG. 6C schematically illustrates a side view of an example stimulation assembly 310 in which the at least one fin 330 comprises a plurality of fins 330 distributed along the longitudinal axis 322 in accordance with certain implementations described herein. Each fin 330 of the plurality of fins 330 extends in the longitudinal direction (e.g., with a length L_{f} along the longitudinal direction in a range of 50 microns to 1 millimeter) and is separated from other adjacent fins 330 by one or more gaps 332 (e.g., with a length L_{g} along the longitudinal direction in a range of 50 microns to 250 microns). In certain implementations, at least one fin 330 of the plurality of fins 330 has dimensions (e.g., length L_{f}, height H; thickness T) that are substantially the same as those of at least one other fin 330 of the plurality of fins 330 (e.g., all the fins 330 having substantially the same length L_{f}, height H, and/or thickness T). In certain implementations, at least one fin 330 of the plurality of fins 330 has one or more dimensions that differ from those of at least one other fin 330 of the plurality of fins 330 (e.g., at least two fins 330 have substantially different lengths L_{f}, heights H, and/or thicknesses T). In certain implementations, the total length L_{F} of the plurality of fins 330 including the gaps 332 is greater than or equal to the length L_{S} of the array of stimulation elements 340, while in certain other implementations, the total length L_{F} of the plurality of fins 330 including the gaps 332 is less than the length L_{S} of the array of stimulation elements 340. In certain implementations, the fins 330 of the plurality of fins 330 are aligned with one another (see, e.g., FIG. 6C), while in certain other implementations, at least one fin 330 of the plurality of fins 330 is at a first azimuthal angle about the longitudinal axis 322 and another at least one fin 330 of the plurality of fins 330 is at a second azimuthal angle about the longitudinal axis 322, the second azimuthal angle different from the first azimuthal angle.

FIG. 6D schematically illustrates a cross-sectional micrograph of a cochlea 140 overlayed with a schematically illustrated example stimulation assembly 310 implanted within the cochlea 140 in accordance with certain implementations described herein. The stimulation assembly 310 is shown to be within the canals 236 of the cochlea 140 with the at least one stimulation electrode 340 facing the modiolar walls of the canals 236 and the at least one fin 330 extending away from the modiolar walls of the canals 236.

In certain implementations, the at least one fin 330 is compressed in a direction substantially parallel to the longitudinal axis 322 when the stimulation assembly 310 is deflected to be substantially straight (e.g., when the stimulation assembly 310 is constrained; within the sheath 260) and the at least one fin 330 provides a restoring force (e.g., in a direction opposite to the straightening deformation of the stimulation assembly 310) configured to counter the straightening of the stimulation assembly 310 and to curve the stimulation assembly 310 (e.g., when the stimulation assembly 310 is not constrained; outside the sheath 260). The dimensions of the at least one fin 330 (e.g., height H; thickness T) can vary along the longitudinal direction such that the stimulation assembly 310 has selected mechanical properties that switch the stimulation assembly 310 from the straightened configuration to the curved configuration in a predetermined manner. In certain implementations, the at least one fin 330 is configured to include one or more portions that are more flexible than other portions of the at least one fin 330 (e.g., the one or more portions providing a smaller restoring force and being easier to straighten than the other portions), thereby providing regions of the stimulation assembly 310 at which the straightening deformation occurs. For example, the at least one fin 330 can comprise a single contiguous fin 330 with the one or more portions comprising holes and/or notches (e.g., having smaller heights H and/or thicknesses T than do the other portions). For another example, the at least one fin 330 can comprise a plurality of fins 330 and the one or more gaps 332 between adjacent fins 330, with the one or more gaps 332 being more flexible than the fins 330.

In certain implementations, as schematically illustrated by the cross-sectional view of FIG. 5F, the at least one fin 330 extends from the outer surface 324 in a single radial direction 326. As shown by FIGS. 5A-5F, the stimulation assembly 310 is curved with the stimulation elements 340 facing a center of curvature of the stimulation assembly 310 and the at least one fin 330 extends radially (e.g., substantially perpendicular to the longitudinal axis 322) and substantially opposite to the directions along which the stimulation elements 340 face.

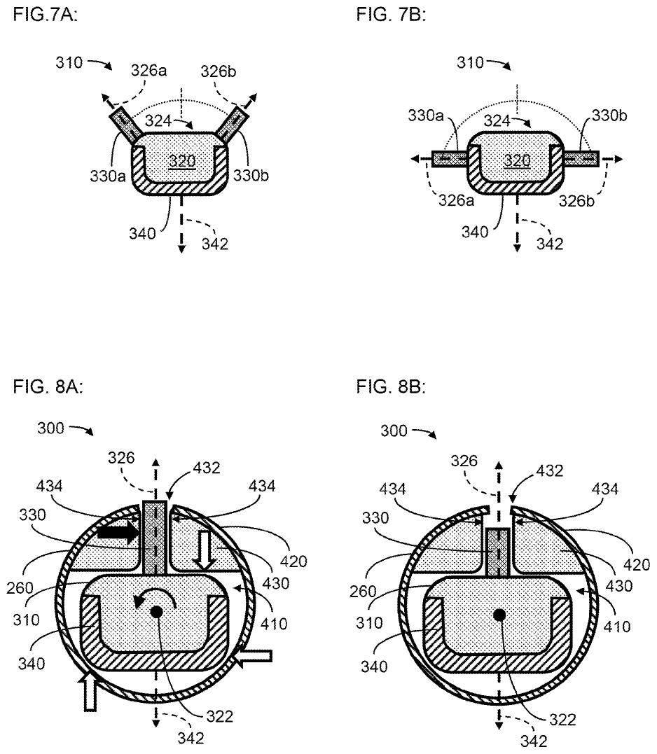

FIGS. 7A and 7B schematically illustrate cross-sectional views in a plane substantially perpendicular to the longitudinal axis 322 of two example stimulation assemblies 310 in accordance with certain implementations described herein. The at least one fin 330 can be located elsewhere on the body 320 besides on the portion of the outer surface 324 directly opposite to the stimulation elements 340. For example, as shown in FIGS. 7A and 7B, the at least one fin 330 can comprise at least one first fin 330a extending from the outer surface 324 in at least one first radial direction 326a and at least one second fin 330b extending from the outer surface 324 in at least one second radial direction 326b (e.g., the first and second fins 330a,b at different azimuthal angles about the longitudinal axis 322). In FIG. 7A, an azimuthal angle difference between the first and second radial directions 326a,b is less than 180 degrees (e.g., substantially equal to 90 degrees), and in FIG. 7B, an azimuthal angle difference between the first and second radial directions 326a,b is substantially equal to 180 degrees (e.g., the at least one first fin 330a and the at least one second fin 330b extending radially in opposite directions). The at least one stimulation element 340 can face at least one third radial direction 342 along a bisector of the angle between the at least one first and at least one second radial directions 326a,b. The example stimulation assembly 310 of FIG. 7B can be utilized in conjunction with lateral wall stimulation electrodes 340.

As shown in FIG. 7A, each of the at least one first fin 330a and the at least one second fin 330b can have a substantial length component that is substantially opposite to the direction 342 along which the stimulation elements 340 face. Upon the stimulation assembly 310 being deflected to be substantially straight (e.g., when the stimulation assembly 310 is constrained; within the sheath 260), the at least one first fin 330a and the at least one second fin 330b of FIG. 7A are at least partially compressed and provide a restoring force that curves the stimulation assembly 310 in a direction 342 along which the stimulation elements 340 face (e.g., when the stimulation assembly 310 is not constrained; outside the sheath 260). In contrast, each of the at least one first fin 330a and the at least one second fin 330b of FIG. 7B have only a relatively small thickness in a direction that is substantially opposite to the direction 342 along which the stimulation elements 340 face, so the at least one first fin 330a and the at least one second fin 330b do not provide a substantial restoring force upon the stimulation assembly 310 being deflected to be substantially straight.

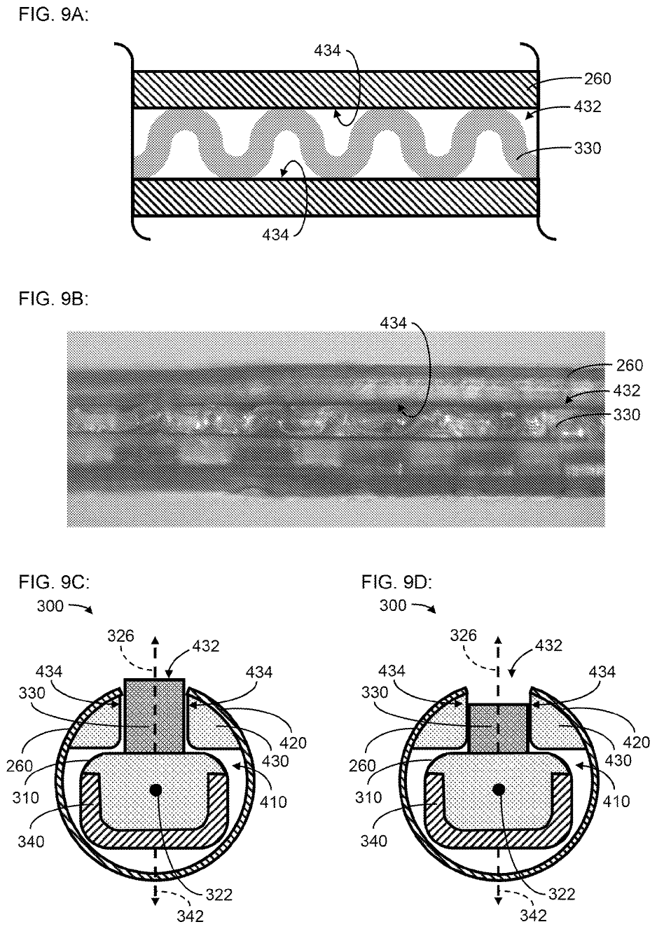

FIGS. 8A and 8B schematically illustrate cross-sectional views in a plane substantially perpendicular to the longitudinal axis 322 of two examples of an apparatus 300 comprising a sheath 260 (e.g., cannula; insertion tube) and a stimulation assembly 310 in accordance with certain implementations described herein. FIG. 9A schematically illustrates a top view of a portion of another example apparatus 300 in accordance with certain implementations described herein. FIG. 9B is a micrograph of a top view of the portion of the example apparatus 300 schematically illustrated by FIG. 9A. FIGS. 9C and 9D schematically illustrate cross-sectional views in a plane substantially perpendicular to the longitudinal axis 322 of two examples of an apparatus 300 comprising a sheath 260 and a stimulation assembly 310 with a buckled fin 330 in accordance with certain implementations described herein.

The stimulation assembly 310 has an unflexed coil shape (e.g., pre-curved; see, e.g., FIGS. 5A-5E, 6A-6D) and comprises the at least one stimulation element 340 facing a first (e.g., radial) direction 342 and at least one fin 330 (e.g., ridge; protrusion; strip) extending along a length of the stimulation assembly 310 (e.g., along the longitudinal axis 322) and in a second (e.g., radial) direction 326 different from the first direction 342 (e.g., opposite to the first direction 342). The sheath 260 comprises an internal volume 410 configured to contain the stimulation assembly 310. The sheath 260 can have a substantially straight shape and the pre-curved stimulation assembly 310 can be flexed within the sheath 260 to have a substantially straight shape within the sheath 260.

In certain implementations, the sheath 260 comprises a tubular (e.g., substantially cylindrical) housing 420 (e.g., comprising a biocompatible metal or polymer material) and at least one structure 430 (e.g., flats comprising a biocompatible metal or polymer material; slit; gap; channel) configured to engage with the at least one fin 330 of the stimulation assembly 310 to resist (e.g., prevent, inhibit, limit, reduce, or minimize) twisting of the stimulation assembly 310 about the longitudinal axis 322. The at least one structure 430 can comprise the same material as the housing 420 or can comprise a stiffer material than the material of the housing 420. The at least one structure 430 can be configured to reduce (e.g., minimize; prevent; inhibit) friction with the body 320 and/or the at least one fin 330 as the stimulation assembly 310 is slid along the sheath 260. For example, the at least one structure 430 can be self-lubricating.

For example, the at least one structure 430 can comprise an elongate slit 432 extending at least partially along the sheath 260, the slit 432 having two opposing surfaces 434 configured to receive the at least one fin 330 while the stimulation assembly 310 is within the inner volume 410. For a pre-curved stimulation assembly 310 implanted via a sheath 260 with an elongate slit 432, the at least one fin 330 can be configured to engage with the slit 432 to enforce the desired orientation of the at least one stimulation electrode 340 (e.g., to prevent twisting of the stimulation assembly 310).

In certain implementations (see, e.g., FIGS. 8A and 9C), the at least one fin 330 has a height H that is sufficiently large to extend through the elongate slit 432 to a region outside the sheath 260 (e.g., beyond the housing 420). In certain other implementations (see, e.g., FIGS. 8B and 9D), the at least one fin 330 does not extend through the elongate slit 432 to a region outside the sheath 260. In certain implementations, the slit 432 has a width (e.g., distance between the two surfaces 434) that is substantially equal to the thickness T of the at least one fin 330, while in certain other implementations, the slit 432 has a width that is substantially greater than the thickness T of the at least one fin 330 (see, e.g., FIGS. 9A-9D).

As shown in FIGS. 8A and 8B, the slit 432 comprises a pair of substantially flat surfaces 434 (e.g., substantially parallel to one another) extending along the at least one fin 330. Upon twisting of the stimulation assembly 310 about the longitudinal axis 322 (denoted in FIG. 8A by the curved arrow), at least one of the surfaces 434 provides a force (denoted in FIG. 8A by a black arrow) on the at least one fin 330 that counteracts the twisting. The stimulation assembly 310 also presses against other portions of the sheath 260 upon the twisting, producing other forces (denoted in FIG. 8A by white arrows) applied to the body 320 and/or the at least one stimulation electrode 340 that also counteract the twisting. In certain implementations, the force from the at least one surface 434 is applied to the at least one fin 330 at a position farther from the centroid of the stimulation assembly 310 than are the other forces applied to the other portions of the stimulation assembly 310. In this way, the at least one structure 430 and the at least one fin 330 can provide a torque that can be more effective in counteracting the twisting of the stimulation assembly 310 than are the other forces at other positions on the stimulation assembly 310.

In certain implementations, the at least one structure 430 is co-extensive with the at least one fin 330 along the longitudinal axis 322 (e.g., the at least one structure 430 provides an anti-twisting torque to the at least one fin 330 over the length L_{F} of the at least one fin 330). In certain other implementations (e.g., in which the width of the stimulation assembly 310 is tapered along the longitudinal axis 322), the at least one structure 430 is only in distal end portion 262 of the sheath 260 from which the stimulation assembly 310 is moved out of the sheath 260 into the recipient's tissue (e.g., the at least one structure 430 provides an anti-twisting torque to the at least one fin 330 over a fraction of the length L_{F} of the at least one fin 330).

In certain implementations, as shown in FIGS. 9A and 9B, the at least one fin 330 is configured to buckle at one or more locations along the length of the stimulation assembly 310 while the stimulation assembly 310 is within the inner volume 410 of the sheath 260. The buckling of the at least one fin 330 is in response to the straightening deformation (e.g., being compressed in a direction substantially parallel to the longitudinal axis 322; when the stimulation assembly 310 is constrained within the sheath 260). As shown in FIGS. 9A and 9B, the fin 330 (e.g., viewable through the slit 432 of the sheath 260) can be buckled at a plurality of locations into a wavy (e.g., serpentine) shape between the surfaces 434 of the slit 432. In certain such implementations, the sheath 260 is configured to accommodate the buckling of the at least one fin 330. For example, as shown in FIGS. 9C and 9D, the slit 432 can have a width that is substantially greater than the thickness T of the at least one fin 330 (e.g., in a range of 2× to 4× the thickness T of the at least one fin 330). While the fins 330 of FIGS. 9C and 9D have the same thickness T as do the fins 330 of FIGS. 8A and 8B, the cross-sectional views of FIGS. 9C and 9D are in a cross-sectional plane in which the buckled fins 330 extend across the width of the slit 432.

In certain implementations, the at least one fin 330 is configured to controllably buckle at predetermined locations along the stimulation assembly 310. The at least one fin 330 can comprise first fin portions distributed along the longitudinal axis 322 and having a first flexibility and second fin portions distributed along the longitudinal axis 322 and having a second flexibility greater than the first flexibility. The second fin portions can be configured to buckle while the stimulation assembly 310 is within the inner volume 410 and the first fin portions can be configured to not buckle while the stimulation assembly 310 is within the inner volume 410. For example, the first fin portions can have a first thickness T₁ in a transverse direction (e.g., substantially perpendicular to the longitudinal axis 322) and the second fin portions can have a second thickness T₂ in the transverse direction, the second thickness T₂ less than the first thickness T₁ (e.g., such that the at least one fin 330 is configured to preferentially buckle at the thinner second fin portions than at the thicker first fin portions). For another example, the first fin portions can extend a first height H₁ in a radial direction (e.g., substantially perpendicular to the longitudinal axis 322) and the second fin portions can have a second height H₂ in the radial direction, the second height H₂ less than the first height H₁ (e.g., such that the at least one fin 330 is configured to preferentially buckle at the shorter second fin portions than at the taller first fin portions).

In certain implementations, once buckling of the at least one fin 330 has occurred, the at least one fin 330 is significantly less able to oppose the compression force resulting from the straightening deformation of the stimulation assembly 310. The dimensions and/or the materials of the body 320 and/or the at least one fin 330 can be selected so that the at least one fin 330 provides a significant contribution to the total bending stiffness of the stimulation assembly 310, and the buckling can result in a significant reduction of the total bending stiffness of the stimulation assembly 310 at large straightening deformations (e.g., when the stimulation assembly 310 is substantially straight). In this way, the stimulation assembly 310 can have less resistance to the straightening deformation and less tendency to twist during straightening. In certain implementations having at least one fin 330 configured to buckle, the stiffness of the sheath 260 sufficient to hold the stimulation assembly 310 in the straightened configuration can be reduced as compared to stimulation assemblies 310 with at least one fin 330 that is not configured to buckle and/or the shape retention (e.g., tightness of curvature) of the stimulation assembly 310 can be increased without increasing the stiffness of the sheath 260.

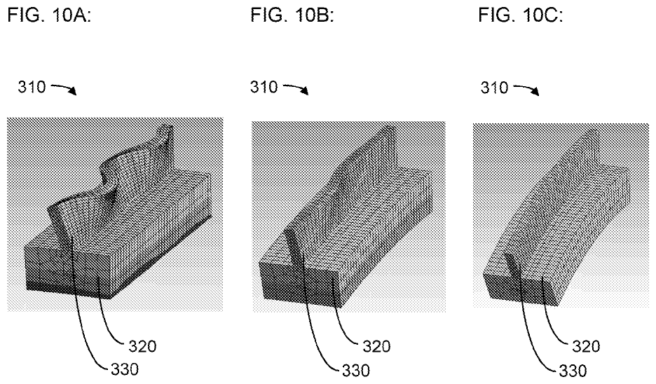

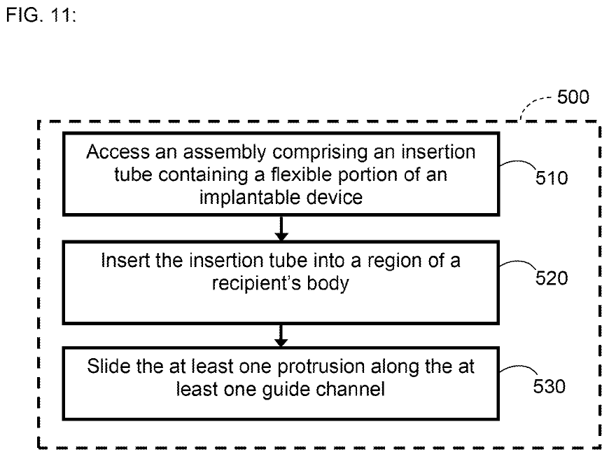

FIGS. 10A-10C schematically illustrate finite element simulations of a portion of an example pre-curved stimulation assembly 310 in various configurations in accordance with certain implementations described herein. The sheath 260 is not shown in FIGS. 10A-10C. As shown in FIG. 10A, the portion of the stimulation assembly 310 within the sheath 260 is deformed to have a substantially straight configuration and the fin 330 is buckled to have a wavy shape. As shown in FIG. 10B, the portion of the stimulation assembly 310 extends partly out of the distal end portion 262 of the sheath 260 and the fin 330 is less buckled. As shown in FIG. 10C, the portion of the stimulation assembly 310 is fully out of the sheath 260 and is able to regain its non-compressed, pre-curved configuration and the fin 330 is not buckled.

FIG. 11 is a flow diagram of an example method 500 in accordance with certain implementations described herein. While the method 500 is described by referring to some of the structures of the example apparatus 300 described herein, other apparatus and systems with other configurations of components can also be used to perform the method 500 in accordance with certain implementations described herein.

In an operational block 510, the method 500 comprises accessing an assembly (e.g., apparatus 300) comprising an insertion tube (e.g., sheath 260) containing a flexible portion (e.g., stimulation assembly 310 comprising an array of stimulation electrodes 340) of an implantable device (e.g., a cochlear implant). The flexible portion has a substantially straight shape within the insertion tube and a coil shape outside the insertion tube. The implantable device comprises at least one protrusion (e.g., fin 330) extending radially outward from the flexible portion. The insertion tube comprises at least one guide channel (e.g., slit 432) extending along the insertion tube and at least partially containing the at least one protrusion. For example, the assembly comprising the insertion tube and the flexible portion of the implantable device can be shipped and/or stored in a hermetically sealed container and said accessing can comprise opening the container and removing the assembly from the container.

In an operational block 520, the method 500 further comprises inserting the insertion tube containing the flexible portion into a region of a recipient's body (e.g., cochlea 140). For example, said inserting can comprise inserting the insertion tube at least partially into a cochlea 140 of the recipient's body. In an operational block 530, the method 500 further comprises sliding the at least one protrusion along the at least one guide channel such that the flexible portion extends out of the insertion tube through a distal end of the insertion tube. For example, said sliding can comprise extending at least a portion of the flexible portion (e.g., a portion of the array of stimulation electrodes 340) into the cochlea 140.

In certain implementations, the at least one guide channel is substantially straight, and the at least one guide channel and the at least one protrusion prevent twisting of the flexible portion within the insertion tube (e.g., twisting about a longitudinal axis 322 of the stimulation assembly 310) during implantation of the implantable device. In certain implementations, the method 500 further comprises removing the insertion tube from the recipient's body after said sliding.

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of conventional cochlear implants, various implementations described herein can be incorporated in a variety of other suitable devices, methods, and contexts. More generally, as can be appreciated, certain implementations described herein can be used in a variety of implantable medical device contexts.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein, but should be defined only in accordance with the claims and their equivalents.

1. An apparatus comprising:
a stimulation assembly configured to be implanted on or within a recipient, the stimulation assembly comprising:
an elongate body having a longitudinal axis;
at least one fin extending in a longitudinal direction substantially parallel to the longitudinal axis and extending from an outer surface of the body; and
at least one stimulation element facing outwardly from the body, the at least one stimulation element configured to be in operative communication with a portion of tissue of the recipient, the at least one fin overlapping at least a portion of the at least one stimulation element.

2. The apparatus of claim 1, wherein the at least one fin extends from the outer surface of the body in at least one radial direction substantially perpendicular to the longitudinal axis.

3. The apparatus of claim 1, further comprising a substantially straight sheath configured to contain the body, the sheath comprising a distal end portion through which the stimulation assembly is configured to be extended out of the sheath.

4. The apparatus of claim 3, wherein the body is sufficiently flexible such that the longitudinal axis of a first portion of the body contained within the sheath is substantially straight and the longitudinal axis of a second portion of the body extended out from the end of the sheath is substantially curved.

5. The apparatus of claim 4, wherein the longitudinal axis of the second portion of the body is curved in a direction in which the at least one stimulation element faces.

6. The apparatus of claim 5, wherein the longitudinal axis of the curved body has a radius of curvature in a range of 2 millimeters to 5 millimeters.

7. The apparatus of claim 3, wherein the sheath comprises at least one structure configured to receive the at least one fin and to allow the at least one fin to be slid along the at least one structure while the stimulation assembly is slid along the sheath.

8. The apparatus of claim 7, wherein the at least one structure and the at least one fin are configured to inhibit twisting of the stimulation assembly about the longitudinal axis.

9. The apparatus of claim 1, wherein the body comprises a first elastomeric material and the at least one fin comprises a second elastomeric material different from the first elastomeric material.

10. The apparatus of claim 1, wherein the at least one stimulation element comprises an array of stimulation elements distributed along the longitudinal axis and the at least one fin comprises a single contiguous fin.

11. The apparatus of claim 10, wherein the single contiguous fin has a length in the longitudinal direction that is less than a length of the array of stimulation elements in the longitudinal direction.

12. The apparatus of claim 1, wherein the at least one stimulation element comprises an array of stimulation elements distributed along the longitudinal axis and the at least one fin comprises a plurality of fins distributed along the longitudinal axis.

13. (canceled)

14. The apparatus of claim 1, wherein the at least one stimulation element comprises a plurality of stimulation elements facing directions towards a center of curvature of the stimulation assembly and the at least one fin extends radially and substantially opposite to the directions along which the stimulation elements face.

15. The apparatus of claim 1, wherein the at least one fin comprises at least one first fin extending from the outer surface in at least one first radial direction and at least one second fin extending from the outer surface in at least one second radial direction, the at least one stimulation element facing at least one third radial direction along a bisector of an angle between the at least one first and the at least one second radial directions.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. An apparatus comprising:
a stimulation assembly having an unflexed coil shape, the stimulation assembly comprising:
at least one stimulation element facing a first direction; and
at least one ridge extending along a length of the stimulation assembly and in a second direction different from the first direction; and
a substantially straight insertion tube comprising an inner volume configured to contain the stimulation assembly flexed to have a substantially straight shape.

35. The apparatus of claim 31, wherein the insertion tube further comprises an elongate slit extending at least partially along the insertion tube, the slit configured to receive the at least one ridge while the stimulation assembly is within the inner volume.

36. The apparatus of claim 31, wherein the at least one ridge is configured to buckle at one or more locations along the length of the stimulation assembly while the stimulation assembly is within the inner volume.

37. The apparatus of claim 31, wherein the at least one ridge comprises first ridge portions having a first flexibility and second ridge portions having a second flexibility greater than the first flexibility, the second ridge portions configured to buckle while the stimulation assembly is within the inner volume and the first ridge portions configured to not buckle while the stimulation assembly is within the inner volume.

38. The apparatus of claim 34, wherein the first ridge portions have a first thickness in a transverse direction, the second ridge portions have a second thickness in the transverse direction, the second thickness less than the first thickness.

39. (canceled)

40. An apparatus comprising:
a stimulation electrode array comprising a body with a pre-curved configuration configured to be flexed into a substantially straight configuration prior to being implanted at least partially within a cochlea of a recipient and to return to the pre-curved configuration upon being implanted within the cochlea, the stimulation electrode array comprising a plurality of stimulation electrodes distributed along a first length of the body and configured to face modiolar wall portions of the cochlea upon being implanted within the cochlea;
at least one fin extending longitudinally along the body and outwardly from the body, the at least one fin configured to extend away from the modiolar wall portions of the cochlea upon being implanted within the cochlea, the at least one fin distributed along a second length of the body, the second length at least partially co-extensive to the first length; and
a substantially straight insertion tube comprising an elongate channel extending longitudinally along the tube, the channel configured to engage with the at least one fin and to allow the at least one fin to be moved along the channel during implantation of the stimulation electrode array within the cochlea, the insertion tube sufficiently rigid to flex the stimulation electrode array into the substantially straight configuration.
